SEQUENCE LISTING (6 sequences)

SEQ ID NO: 1 - DNA, 2245 bp, Zea mays
  CDS (138)..(1844)
cgaaaaccctcgcggcgaggagcgctctccgtcccacacacgcgcgccgcagcctccgcagtccgcacctgcctccacgacctgtcgtgctcgtgcgctactctcctcgagcgcggcgcggagcacggggacatcaaatggcggcagcggcggagatcggtggtttcgcggcggcgagagtggcggtggcaacgctcaggccggctgcgcacccagccccggcggcggcggcggcaccgcagcctaggagggcggtggcggcgcagtcgctgcagacgaccgccaccgaggcgctgacggcggatctcgccggcacgaccaacggcgctgtgcatgctcggatgaatagtaaggctacaagtgaaatcacttcacaggcagttactgcaaattctaggagaaagacaaagatagtctgcaccataggtccctcaaccaacactcgtgagatgatttggaagcttgcagagactggaatgaatgtagcgcgccttaatatgtcccatggtgaccaccagtcgcaccagaaggttattgatttggtcaaggagtacaatgcacagaacactgatggcaatgttattgccattatgctggacacaaagggtcctgaagttagaagtggggatgttccagagccaatcatgctcaaggaaggtcaagagttcaacttcacgattaaaagaggggtgagcactgaagacactgtcagcgtgaactatgatgacttcataaatgatgttgaagctggcgacatactattagtggatggaggaatgatgtcgcttgctgtgaagtctaaaacagccgatacagtcaagtgtaaagtagttgatggtggggaattgaaatcacggcgccacctaaatgtccgtggaaagagtgctactttgccatctatcactgagaaggattgggaagacataaaatttggtgtcgaaaacggtgttgatttctatgcagtttcctttgtgaaggatgccaaagttatccatgaattaaaagactaccttaaaagtgctaatgccgatatacatgtcattccaaaaattgaaagtgcagattcaataccaaacctgcagtccattattgctgcttcagatggggcaatggtggcgcgtggagaccttggtgctgaacttccgattgaggatgttcctttgctacaggcagagattgtccaaacatgtcgaagcatggagaaaccagtcattgtcgctacaaatatgttggaaagcatgattgaccatcctactcccactagggcagaagtttctgacatagctattgcagttcgggaaggtgctgatgccatcatgttatctggcgaaactgctcatggaaagtatccactaaaggcagtcaaggtgatgcacactgtggcactcagaacagaatccagcctttataacccaactacttctcctagtcttgttgcatctgcacagggtctacagaatgaggacttctccgcaagccagctaagtaaaatgttcggatctcatgcaacgatgatggccaacacccttcgcacaccaatcattgtatttacacagacaggctccatggctgtcctcctgagccactatcgtccctcgtctacactatttgcatttacaaacgaggaacgagtgaagcaacggctagcactctaccagggcgtcatccctattcacatgcagttctctgacgacgcagaagaaactttctccagagcaattagcagcttgctgaaagcacaatatgtgaagaagggagactacgtcactcttgttcagagcggagtgacttcaatctggagagaggaatccactcaccacatccaagtgaggaaagttcaggtctgatgtgccggtgggaattggtcgtctgagaaattttgatagcgccgcctgatgtgttatcatcattatatgtgtaattttactgttttaccaggagattgctacgtcgagttatatgttgtgtcgaattcacgtgtaggctctgaatcttgactgtgttcgttcattttcgcttgtttcacactgaagtgttataagctcaactttactgcttttgttttcttgtgaaacttgagtttagtttcttgttacaaaaggagctagcactaacagggtggtgagttttgtaaacggggcgaggactgttgagttaaaactgttgtaataatgatatcgttcctgcaagacgacacatgcatggccagaaggccaaagtcaaaaaaaaaaaaaaaaaaaaaaaa

SEQ ID NO: 2 - protein, 569 aa, Zea mays
MAAAAEIGGFAAARVAVATLRPAAHPAPAAAAAPQPRRAVAAQSLQTTATEALTADLAGTTNGAVHARMNSKATSEITSQAVTANSRRKTKIVCTIGPSTNTREMIWKLAETGMNVARLNMSHGDHQSHQKVIDLVKEYNAQNTDGNVIAIMLDTKGPEVRSGDVPEPIMLKEGQEFNFTIKRGVSTEDTVSVNYDDFINDVEAGDILLVDGGMMSLAVKSKTADTVKCKVVDGGELKSRRHLNVRGKSATLPSITEKDWEDIKFGVENGVDFYAVSFVKDAKVIHELKDYLKSANADIHVIPKIESADSIPNLQSIIAASDGAMVARGDLGAELPIEDVPLLQAEIVQTCRSMEKPVIVATNMLESMIDHPTPTRAEVSDIAIAVREGADAIMLSGETAHGKYPLKAVKVMHTVALRTESSLYNPTTSPSLVASAQGLQNEDFSASQLSKMFGSHATMMANTLRTPIIVFTQTGSMAVLLSHYRPSSTLFAFTNEERVKQRLALYQGVIPIHMQFSDDAEETFSRAISSLLKAQYVKKGDYVTLVQSGVTSIWREESTHHIQVRKVQV

SEQ ID NO: 3 - DNA, 1922 bp, Zea mays
  CDS (148)..(1674)
cccggggaagaatccttctcgacgccgcccccattcattcttggagggcaagaagaacgcgcagtccaaaagcgaaacaaggggaaggggcgaggcagccaaacgaatcccagcacctgcctctcctttgcttgcgcagatctagcgatggcggcgggcggtgagttggcgtggggggaggagcccgtggcgcggcgccggcctaagaccaagatcgtctgcacgctcggcccggcgtcgcgatccgtcgagatgatctcgcgcctgctgcgcgccgggatgtgcgtcgcgcgcttcaacttctcccatggctcccacgagtaccaccaggagacgctcgacaacctgcacgccgccatggagctcaccgggatcctctgcgccgttatgctcgacaccaagggtccagagattagaactggatttttgaaagatgggaagcctattcaattgataaaaggccaagaaatcacaatttctacagactatagcattcagggtgatgagaaaatgatatcaatgagctacaagaagcttgctgttgatctgaagccaggcagtgtcatattgtgtgctgatggtaccatcactcttactgttcttcactgtgataaagaacaaggcttggttcgctgccgctgtgagaacactttcaagcttggcgagaggaagaatgttaatcttccaggagttattgttgatctccccacactcacagacaaggacaaggaggacatccttaaatggggtgttccaaacaagattgacatgattgccttgtcatttgttcggaagggttcggatcttgtggaggttaggaaggtacttggtgagcatgccaagtccataatgctgatgtcaaaggttgagaatcaagagggagtagctaactttgatgatatcctggcaaactctgatgcttttatggtggcaagaggtgatttaggaatggaaattcctatagagaagattttctttgcacagaaggtgatgattttcaagtgcaatattcaaggcaagcctgttgtgactgcaacccagatgttggaatctatgatcaagtctcctcgccctactcgggcagaagcaacggatgttgcaaacgcagttcttgatggcacagactgtgttatgctcagtggtgagactgctgctggggcttatccagggttggcggtgcagactatggctaagatctgcctgcaagcagagtcttgcgtagaccatgctgctattttcaaatcaatcatggcttcagctccaattccaatgagtccattggagagccttgcatcatcagctgttcgcacagcaaactctgccagggcagcacttatcttggtcctgaccaggggaggaactactgctaggcttgtagccaagtataggccatccatgccgattctgtccgttgtggttcctgagctgaagacagactcgttcgactgggcctgcagtgacgagggtccggcaaggcacagcctcattgtgaggggtgtcatcccaatgctgagtgccgccactgccaaggcctttgacaacgaagccactgacgaagctattggttttgccattgaaaatgcgaagacgatggggctatgcaacaccggtcaatccgttgtggccctgcaccgcatcgggatttcatccgtcatcaagcttcttactgtgaagtaggggtccaagggtgagggacaccattttgcttggtgaatttttgtgaggctgcttacgcacatcagcgtatccacagcttttctgttgccaaagatacagactttgcgttctggtacaccttggtgttacaaggttgtgggaatttgtcgtttagatattagcgatttaaacagtttatcaactgctatagctaattaatttgcagctgtgctgcaactttatcgtactgaaaaaaaaaaaaaaaa

SEQ ID NO: 4 - protein, 509 aa, Zea mays
MAAGGELAWGEEPVARRRPKTKIVCTLGPASRSVEMISRLLRAGMCVARFNFSHGSHEYHQETLDNLHAAMELTGILCAVMLDTKGPEIRTGFLKDGKPIQLIKGQEITISTDYSIQGDEKMISMSYKKLAVDLKPGSVILCADGTITLTVLHCDKEQGLVRCRCENTFKLGERKNVNLPGVIVDLPTLTDKDKEDILKWGVPNKIDMIALSFVRKGSDLVEVRKVLGEHAKSIMLMSKVENQEGVANFDDILANSDAFMVARGDLGMEIPIEKIFFAQKVMIFKCNIQGKPVVTATQMLESMIKSPRPTRAEATDVANAVLDGTDCVMLSGETAAGAYPGLAVQTMAKICLQAESCVDHAAIFKSIMASAPIPMSPLESLASSAVRTANSARAALILVLTRGGTTARLVAKYRPSMPILSVVVPELKTDSFDWACSDEGPARHSLIVRGVIPMLSAATAKAFDNEATDEAIGFAIENAKTMGLCNTGQSVVALHRIGISSVIKLLTVK

SEQ ID NO: 5 - DNA, 2075 bp, Zea mays
  CDS (198)..(1736)
gcgcccacggaatcgcaattccccactcccaccccccttctcctttctataaagggaaacttcgccaccaaatctcgaatcccatctcgctgctctccgccgctcgacgccgacgccgacgccgacacctctccctcccccattccccgtgccccggaccgcggagcagacagaggcgagatccggagcgccgcgcgatggcgaacatcgacatggcgaagatcctggcggacctggaccgcggcgccggcgacgcgcggctgcccaagaccaagctcgtctgcacgcttggcccggcctcccgctccgtgcccatgctcgagaagctgctccgcgccggcatgaacgtcgcgcgcttcaacttctcccacggcacccaccagtaccaccaggagacgctcgacagcctccgccaggccatgcacaacaccggcatcctctgcgccgtcatgctcgacaccaagggtcctgagattcgtacagggtttttgaaggatggtaaaccaattaagctaaccaagggtcaagagatcactgttaccactgattatgatatcaagggtgacgaaaaaatgatcgctatgagttacaagaaactgcctgttgacgtgaagcctggaaatgtcatactctgcgcagatggtactatctctctggccgttctgtcttgcgatcctgatgctggaactgtgcgttgtaggtgtgagaacactgcaatgcttggtgagagaaagaactgcaatttgccaggaattgttgtggatcttcctacattgactgagaaagataaagaagatattttgggctggggtgttccaaatgacattgacatgattgctctatcctttgtccgtaaaggatcagatttggtgactgtcagacaggttcttgggcagcatgccaagcgcattaagttgatgtcaaaggttgagaaccaagagggtgttgtaaactttgatgagatcttgagggagactgatgcttttatggttgctagaggtgatctgggaatggagattccagtcgagaagattttccttgcgcagaagatgatgatctacaagtgcaacattgctggcaaacctgttgttactgctacccagatgctcgagtcgatgatcaaatctcctcggccgacacgtgctgaggccactgacgttgcaaatgccgttcttgatggaactgactgtgtcatgctcagcggagagagtgccgctggagcctaccccgaggtggctgtgaagatcatggctcgtatttgcatcgaggcagaatcttccctggaccacgaggcagttttcaaggccatgatcaggtccgcgcctcttccaatgagccctctggagtcccttgcatcatctgctgtgaggactgccaacaaggccaaggctgcactgattgtcgtcctgactcgcggtggcaccacggccaaactggtcgccaagtaccgccccagggtccccatcctctccgttgtcgtacccgtgcttacgaccgactctttcgactggaccatcagctccgagggcccagctaggcacagcctgatctacagaggcctcattcccctcctcgccgagggctccgcgaaggctacggattcagagtcgacggaggtgatccttgaggccgcgttgaagtcggccgtgcagaagcagctctgcaagcctggtgatgccatcgtggctcttcatcgtattggcgtcgcctccgtcatcaagatctgcatcgtgaagtgatcaccacgacctggttctcactgctgggattccatgaaacagcttttctgcctcctttttactgcatttttttccggagacagatatttatgttgtaagcctgcgtctgttgtttgcagttgcggaatgttttttgttcacgtagttatttagctggtaaatggggagggagaggactccattgatgttagtggtaataagatttgtgtctatggagttgagttgaaagggttttttcatgctaagccccgagacactaggagatggagatcgaggttgagatgagataagcaataagtgaaaaaaatgttgatttgcaaaaaaaaaaaaaaaaaa

SEQ ID NO: 6 - protein, 513 aa, Zea mays
MANIDMAKILADLDRGAGDARLPKTKLVCTLGPASRSVPMLEKLLRAGMNVARFNFSHGTHQYHQETLDSLRQAMHNTGILCAVMLDTKGPEIRTGFLKDGKPIKLTKGQEITVTTDYDIKGDEKMIAMSYKKLPVDVKPGNVILCADGTISLAVLSCDPDAGTVRCRCENTAMLGERKNCNLPGIVVDLPTLTEKDKEDILGWGVPNDIDMIALSFVRKGSDLVTVRQVLGQHAKRIKLMSKVENQEGVVNFDEILRETDAFMVARGDLGMEIPVEKIFLAQKMMIYKCNIAGKPVVTATQMLESMIKSPRPTRAEATDVANAVLDGTDCVMLSGESAAGAYPEVAVKIMARICIEAESSLDHEAVFKAMIRSAPLPMSPLESLASSAVRTANKAKAALIVVLTRGGTTAKLVAKYRPRVPILSVVVPVLTTDSFDWTISSEGPARHSLIYRGLIPLLAEGSAKATDSESTEVILEAALKSAVQKQLCKPGDAIVALHRIGVASVIKICIVK

(19) United States
(12) Patent Application Publication
(10) Pub. No.: US 2004/0019931 A1
(43) Pub. Date: Jan. 29, 2004

(54) Methods and compositions for modifying oil and protein content in plants

(75) Inventors: Mitchell C. Tarczynski, West Des Moines, IA (US); Changjiang Li, Urbandale, IA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Pioneer Hi-Bred International, Inc.

(21) Appl. No.: 09/930,906

(22) Filed: Aug. 16, 2001

Related U.S. Application Data
(60) Provisional application No. 60/226,142, filed on Aug. 18, 2000.

(51) Int. Cl. A01H 1/00; C12N 9/12; C07H 21/04; C12N 15/82; C12N 5/04

(52) U.S. Cl. 800/281; 435/69.1; 435/194; 435/320.1; 435/419; 536/23.2

(57) ABSTRACT

Compositions and methods for altering pyruvate synthesis in plants are provided. The compositions and methods find use in increasing the oil or protein content of plant tissues, particularly seeds. Novel isolated nucleotide molecules encoding cytosolic and plastidic pyruvate kinases are provided. The methods involve introducing into a plant nucleotide sequences encoding enzymes that alter pyruvate synthesis. Additionally provided are isolated pyruvate kinases and transformed plants, plant tissues, plant cells, and seeds thereof.

### CROSS-REFERENCE TO RELATED APPLICATIONS

This application claims the benefit of U.S. Provisional Application No. 60/226,142, filed Aug. 18, 2000, which is hereby incorporated herein in its entirety by reference.

### FIELD OF THE INVENTION

The invention relates to plant metabolism, particularly to genes encoding metabolic enzymes and to the transformation of plants with such genes.

### BACKGROUND OF THE INVENTION

A major focus of crop plant breeders over the last fifty years has been the modification of traits that affect seed composition. Of particular interest to plant breeders have been traits that affect the energy reserves of the seed, particularly oil, protein, and starch. The efforts of plant breeders have led to the development and introduction of crop plants with modified levels of oil, protein, and starch. Additionally, plant breeders have developed cultivars with modified oil, protein, and starch compositions. For example, plant breeders have developed low-erucic-acid varieties of rapeseed (Brassica sp.) and high-lysine corn. Because the most important components of seeds that are used for human and livestock consumption are oil, protein, and starch, crop improvement efforts will continue to focus on these components.

While mankind has derived many benefits from the past efforts of plant breeders, the combination of the rapidly increasing human population and the decline in land available for agriculture places a tremendous burden on agriculturists to increase both agricultural output and productivity. New and improved crop plant varieties are desired by agriculturists to help meet the challenge of feeding the world's human population. Although traditional plant breeding approaches for crop plant improvement have been successful, the traditional approaches are slow and limited to naturally occurring genetic variation or artificially induced mutations. To keep pace with the escalating demands that the increase in the world's population places on agriculture, more rapid approaches for developing crop plants are necessary. The recombinant-DNA-based methodologies of genetic engineering have already been used successfully to incorporate new insect-resistance and herbicide-tolerance traits into crop plants. Such methodologies have great potential for modifying other characteristics of crop plants including the starch, oil, and protein levels in seeds.

### SUMMARY OF THE INVENTION

Methods and compositions are provided for altering the synthesis of pyruvate in plants. The compositions comprise nucleotide molecules comprising nucleotide sequences encoding plastidic and cytosolic forms of pyruvate kinase. The nucleotide molecules of the invention are useful in transforming plants for tissue-preferred, seed-preferred, and constitutive expression of pyruvate kinase. The compositions find use in methods for modifying the protein and/or oil content of a plant, or at least one part thereof. The methods involve introducing into a plant at least one nucleotide construct comprising a nucleotide sequence encoding a pyruvate kinase. The methods can further comprise introducing into a plant at least one additional nucleotide construct comprising a nucleotide sequence encoding malic enzyme or any other nucleotide sequence that encodes an enzyme or protein that is capable of increasing or decreasing the synthesis, or level, of pyruvate in a plant. The methods of the invention find use in increasing the synthesis of protein, amino acids, oil, intermediates in oil synthesis, and specialty molecules in plants.

Expression cassettes comprising nucleotide sequences of the invention are provided. Additionally provided are transformed plants, plant tissues, plant cells, and seeds thereof. Isolated proteins encoded by the nucleotide sequences of the invention are also provided.

### DETAILED DESCRIPTION OF THE INVENTION

The present invention is drawn to compositions and methods for improving agronomic traits in plants. The invention provides compositions and methods that find use in modifying intermediary metabolism in plants. The methods find further use in modifying the oil and protein content of plant tissues, particularly storage tissues such as, for example, seeds. The compositions comprise isolated nucleotide molecules comprising nucleotide sequences encoding cytosolic and plastidic forms of pyruvate kinase and the isolated proteins encoded by such nucleotide sequences. The methods involve altering metabolic processes in a plant to modify intermediary metabolism therein. To alter metabolic processes, plants can be transformed with a pyruvate kinase nucleotide sequence, an NADP⁺-dependent malic enzyme nucleotide sequence, an NAD⁺-dependent malic enzyme nucleotide sequence, or any combination thereof. The methods of the invention find use in agriculture, particularly in the development of plant varieties with high oil and/or high protein seeds and in increasing the levels of desired intermediates or specialty molecules produced in a plant. Such specialty molecules include, for example, the biodegradable, plastic-like polymers known as the polyhydroxyalkanoates (PHA). See, for example, WO 01/23596, herein incorporated by reference.

The invention involves altering metabolic processes in plants. In particular, the invention provides methods and compositions for altering the metabolism of pyruvate in plants. By “altering the metabolism of pyruvate” is intended altering the capability of the plant, or at least one part thereof, to metabolize pyruvate including, but not limited to, increasing or decreasing: the rate of pyruvate synthesis; the level of an enzyme that catalyzes the formation of pyruvate; the activity of an enzyme that catalyzes the formation of pyruvate; the rate of pyruvate degradation or conversion into other molecules; the level of an enzyme that catalyzes the degradation or conversion of pyruvate into other molecules; and the activity of an enzyme that catalyzes the degradation or conversion of pyruvate into other molecules.

The methods of the invention do not, however, depend on any change in the level of pyruvate in a plant cell, or compartment thereof. It is recognized that increasing the synthesis of pyruvate in the cytosol or plastids of a plant cell might not result in increased accumulation of pyruvate in the plant cell, or a compartment thereof. Any additional pyruvate that is formed in the cytosol can be, for example, shunted to mitochondria for entry into the TCA cycle without a detectable change in the pool size of pyruvate in the cytosol. Similarly, any additional pyruvate that is formed in plastids can be, for example, shunted to fatty acid synthesis without a detectable change in the pool size of pyruvate in the plastid.

The invention involves altering the metabolism of pyruvate in specific compartments of plant cells. In a first aspect, the invention involves increasing the synthesis of pyruvate in the cytosol. Pyruvate can be synthesized from phosphoenolpyruvate (PEP) and ADP in an enzymatic reaction catalyzed by a cytosolic form of pyruvate kinase (see [1] below).

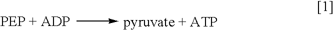

In a second aspect, the invention involves increasing the synthesis of pyruvate in the plastids. Pyruvate can be synthesized in the plastid from PEP and ADP in an enzymatic reaction catalyzed by a plastidic form of pyruvate kinase. In addition, pyruvate can be formed in the plastid from malate and NADP⁺ in a reaction catalyzed by NAD⁺-dependent malic enzyme (see [2] below).

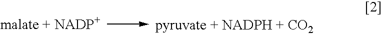

Pyruvate metabolism can be altered in a single compartment within a plant cell or in multiple compartments. Pyruvate metabolism can be altered in any compartment of a plant cell in which pyruvate is known to occur such as, for example, the cytosol or cytoplasm, plastids, mitochondria, and the vacuole. Plastids include, but are not limited to, proplastids, etioplasts, chloroplasts, chromoplasts, leucoplasts, and amyloplasts.

As described above, PEP is a precursor for pyruvate synthesis in both the cytosol and the plastids. PEP is also a key intermediate in the partitioning of carbon between the cytsol and plastids. Like pyruvate, PEP can be synthesized in both the cytosol and plastids. See Plaxton (1996) Annu. Rev. Plant Physiol. Plant Mol. Biol. 47: 185-214, herein incorporated by reference. In the cytosol, PEP is synthesized in the main pathway of carbohydrate catabolism in plants, glycolysis. PEP is the last intermediate formed in glycolysis prior to the synthesis of the end product, pyruvate. In the plastids, PEP is synthesized from 2-phosphoglyceric acid. PEP, that is either synthesized in the plastid or translocated there, can be converted to pyruvate in a reaction catalyzed by plastidic pyruvate kinase as described above.

Additional pyruvate can be synthesized in the plastids from malate. Malate can be synthesized in the cytosol from PEP in two steps. In the first step PEP is converted to oxaloacetate (OAA) (see [3] below). In the second step OAA is converted to malate (see [4] below). Malate can then enter the plastid. Once in the plastid, malate can be converted to pyruvate in an enzymatic reaction involving NADP⁺-dependent malic enzyme as described above.

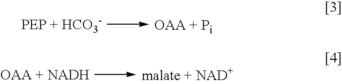

Altering pyruvate metabolism in a plant cell can have profound effects on other metabolic processes in the cell, particularly on the synthesis of amino acids, proteins, fatty acids, triglycerides, and intermediates thereof. Pyruvate that is formed in the cytosol can enter the mitochondrial matrix and can be converted to acetyl-CoA for entry into the TCA cycle. While an essential function of the TCA cycle is ATP production via oxidative phosphorylation, the TCA cycle is also a source of carbon skeletons that are necessary for the synthesis of amino acids. In plants, at least one of pyruvate and the TCA intermediates, α-ketoglutarate and oxaloacetate, is necessary for the biosynthesis of the amino acids, alanine, leucine, valine, glutamic acid, glutamine, arginine, proline, asparatic acid, asparagine, threonine, isoleucine, lysine, and methionine (Heldt (1997) Plant Biochemistry & Molecular Biology, Oxford University Press, New York p. 259). Thus, altering the metabolism of pyruvate in the cytosol can alter the rate of synthesis of amino acids.

Because protein synthesis requires amino acids, an alteration in the rate of synthesis and/or levels of amino acids in a plant can affect protein synthesis. Therefore, altering pyruvate kinase in the cytosol of a cell can lead to changes in the rates of synthesis of both amino acids and proteins in a plant, or part thereof. In seeds, for example, increasing the rate of protein synthesis during seed development can increase the level of protein in the mature seed. Conversely, decreasing the rate of protein synthesis in a developing seed can decrease the level of protein in the mature seed.

Pyruvate that is present in the plastids can the enter the pathway for the biosynthesis of fatty acids. In plants, the de novo synthesis of fatty acids occurs in the plastids. Both saturated and monounsatatured fatty acids up to a carbon-chain length of eighteen are produced in the plastids. Further elongation and desaturation of fatty acids occurs in the endoplasmic reticulum. By altering the synthesis of pyruvate in the plastids, the synthesis of fatty acids can be affected. Decreased production of pyruvate in plastids can lead to lower rates of fatty acid synthesis. In tissues that accumulate oil or triglycerides, decreasing the rate of fatty acid synthesis during seed development can decrease the level of oil in the mature seed. Conversely, increasing the rate of fatty acid synthesis during seed development can increase the level of oil in the mature seed.

Methods are provided for altering the metabolism of pyruvate in the cytosol. The methods find use in increasing the synthesis of amino acids and/or proteins in a plant, or part thereof. In particular, the methods find use in the production of improved cultivars of crop plants having seeds with increased levels of protein. The methods involve introducing into a plant a nucleotide construct comprising at least a portion of a nucleotide sequence encoding a pyruvate kinase. The methods do not depend on a particular nucleotide sequence encoding a pyruvate kinase, only that such a pyruvate kinase nucleotide sequence is capable of affecting pyruvate metabolism in the cytosol. Any nucleotide sequence that is known in the art that encodes a pyruvate kinase sequence can be employed, such as, for example, pyruvate kinase nucleotide sequences that encode pyruvate kinases that are targeted to the cytosol and pyruvate kinase nucleotide sequences that encode pyruvate kinases that are targeted to plastids. The cytosolic pyruvate kinase nucleotide sequences of the invention are not limited to naturally occurring nucleotide sequences, but also include modified sequences. It is recognized that the nucleotide sequence encoding a nuclear-encoded, plastidic pyruvate kinase can be modified for expression of the encoded enzyme in the cytosol by removing the portion of the nucleotide sequence that encodes the chloroplast transit peptide. Cytosolic pyruvate kinase nucleotide sequences of the invention include, but are not limited to, EMBL Accession Nos. Z29492 and X53688, GenBank Accession No. L08632, and the nucleotide sequences set forth in SEQ ID NOs: 3 and 5.

If increased expression of pyruvate kinase is desired, the nucleotide construct will additionally comprise an operable linked promoter that drives expression in a plant cell. Promoters of the invention include, but are not limited to, seed-preferred, constitutive, developmentally regulated, and chemical-regulated promoters.

If a decreased level or activity of pyruvate kinase is desired in the cytosol, the methods of the invention can additionally comprise antisense suppression, co-suppression, and chimeraplasty. Such methods are known in the art. In antisense suppression methods, a pyruvate kinase nucleotide sequence can be operably linked to a promoter that drives expression in a plant for the production of antisense transcripts. In co-suppression methods, a pyruvate kinase nucleotide sequence can be operably linked to a promoter that drives expression in a plant for the production of sense transcripts. With chimeraplasty, no promoter is necessary; only a pyruvate kinase nucleotide sequence, or portion thereof is used.

If desired, one or more additional nucleotide constructs may be introduced into the plant. Such additional nucleotide constructs can comprise, for example, at least a portion of a nucleotide sequence of an enzyme, or other protein, that is capable of affecting the synthesis of pyruvate in the cytosol. If expression of the nucleotide sequence is desired, the nucleotide construct can additionally comprise an operably linked promoter. The invention does not depend on a particular nucleotide sequence. Any nucleotide sequence originating from a gene or transcript that encodes an enzyme or protein that is capable of increasing or decreasing the synthesis and/or level of pyruvate in the cytosol can be employed. Typically, such an enzyme or protein is capable of altering the amount of PEP that is available in the cytosol for the formation of pyruvate. If a decreased level or activity of such an enzyme or protein is desired, the methods of the invention may additionally comprise anti-sense suppression, co-suppression, or chimeraplasty.

In certain embodiments of the invention, the additional nucleotide constructs can comprise, for example, at least a portion of a pyruvate kinase nucleotide sequence or a malic enzyme nucleotide sequence. Such pyruvate kinase nucleotide sequences can encode plastidic pyruvate kinases. Plastidic pyruvate kinase nucleotide sequences of the invention include, but are not limited to, EMBL Accession No. Z28374 and the nucleotide sequence set forth in SEQ ID NO: 1. The malic enzyme nucleotide sequences of the invention include, but are not limited to, those that encode NADP⁺-dependent malic enzyme. Such NADP⁺-dependent malic enzyme nucleotide sequences include, but are not limited to, GenBank Accession Nos. J05130, AB016804, AW217913, and AI727829.

The methods of the invention involve targeting enzymes or proteins to specific cellular compartments such as, for example, the cytosol and the plastids. It is recognized that the localization of a nuclear-encoded protein within the cell is determined by the amino acid sequence of the protein. It is further recognized that the localization of an enzyme or protein can be altered by modifying the nucleotide sequence that encodes the protein in such a manner as to alter the amino acid sequence of the protein. The nucleotide sequences of the invention can be altered to redirect the cellular localization of the encoded proteins by any methods known in the art. Modifications include, but are not limited to, additions, deletions, and substitutions. For example, a cytosolic protein can be redirected to the plastid by operably linking a nucleotide sequence encoding a plastid transit peptide to the nucleotide sequence encoding a cytosolic protein. A nuclear-encoded plastidic protein can be redirected to the cytosol by deleting the nucleotide sequences encoding the transit peptide.

Alternatively, the enzymes and proteins of the invention can be localized to the plastid by transforming the plastid genome with a nucleotide sequence encoding the desired protein. Methods for transforming plastids and cholorplasts are known in the art. Methods for modifying the nucleotide sequence for transcription and translation in plastids are also known in the art.

In a first embodiment of the invention, the methods of the invention provide a plant, particularly a maize plant, that produces seeds or kernels with an increased level of protein. Such seeds can have an increased level of protein in the tissues of the embryo, endosperm, or both. The methods involve stably incorporating into the genome of a plant a nucleotide construct comprising a pyruvate kinase nucleotide sequence, particularly a nucleotide sequence encoding a cytosolic pyruvate kinase, which is operably linked to a promoter that drives expression in a developing seed. The pyruvate kinase nucleotide sequences of the first embodiment include, but are not limited to, the nucleotide sequences set forth in SEQ ID NOs: 3 and 5. Promoters include, for example, those that direct expression preferentially in seeds including, but not limited, to the oleosin and globulin-1 promoters.

Methods are provided for altering the metabolism of pyruvate in plastids. The methods find use in increasing the synthesis of fatty acids and/or oil in a plant, or part thereof. In particular, the methods find use in the production of improved cultivars of crop plants having seeds with increased levels of oil. By increasing the flux of carbon toward oil biosynthesis, increased levels of oil, fatty acids, oil precursors, and specialty molecules that are synthesized from oil precursors can be achieved in a plant, or part thereof. The methods involve introducing into a plant at least one nucleotide construct comprising at least a portion of a nucleotide sequence encoding a pyruvate kinase or an NADP⁺-dependent malic enzyme. In some embodiments of the invention, both a pyruvate kinase nucleotide sequence and a NADP⁺-dependent malic enzyme nucleotide sequence are introduced into the plant. Such nucleotide sequences can be introduced into the plant as a single nucleotide molecule comprising both the pyruvate kinase and a NADP⁺-dependent malic enzyme nucleotide sequences. Alternatively, the pyruvate kinase and a NADP⁺-dependent malic enzyme nucleotide sequences can be introduced into the plant on two separate nucleotide molecules.

The methods of the invention do not depend on a particular nucleotide sequence encoding a pyruvate kinase. Any nucleotide sequence known in the art that encodes a pyruvate kinase sequence that is capable of catalyzing the formation of pyruvate from PEP and ATP in the plastids of a plant cell can be employed. The pyruvate kinase nucleotide sequences of the invention can encode pyruvate kinases that are known to occur in plastids. More preferred pyruvate kinases nucleotide sequences are EMBL Accession No. Z28374 and the nucleotide sequence set forth in SEQ ID NO: 1.

The methods of the invention do not depend on a particular nucleotide sequence encoding NADP⁺-dependent malic enzyme. NADP⁺-dependent malic enzyme nucleotide sequences of the invention include, but are not limited to, GenBank Accession Nos. J05130, AB016804, AW217913, and AI727829.

If increased expression of pyruvate kinase or NADP⁺-dependent malic enzyme is desired in the plastid, the nucleotide construct will additionally comprise an operably linked promoter that drives expression in a plant cell. Such promoters include, but are not limited to, seed-preferred, constitutive, developmentally regulated, and chemical-regulated promoters. If a decreased level or activity of pyruvate kinase or NADP⁺-dependent malic enzyme is desired, the methods of the invention may additionally comprise anti-sense suppression, co-suppression, and chimeraplasty.

If desired, one or more additional nucleotide constructs may be introduced into the plant. Such additional nucleotide constructs can comprise, for example, at least a portion of a nucleotide sequence encoding an enzyme, or other protein, that is capable of affecting the synthesis or the level of pyruvate in the plastid. If expression of the nucleotide sequence is desired, the nucleotide construct can additionally comprise an operably linked promoter. The invention does not depend on a particular nucleotide sequence. Any nucleotide sequence that encodes an enzyme, or protein, that is capable of increasing or decreasing the synthesis of pyruvate in the cytosol can be employed. Typically, such an enzyme or protein is capable of altering the amount of PEP, malate, or both that is available for the formation of pyruvate within the plastid. Such an enzyme can be localized in any compartment or organelle of the cell, including, but not limited to, the cytosol, plastids, mitochondria, and vacuoles. If a decreased level or activity of such an enzyme or protein is desired, the methods of the invention may additionally comprise anti-sense suppression, co-suppression, chimeraplasty, and dominant-negative strategies.

In certain embodiments of the invention, the additional nucleotide constructs can comprise, for example, at least a portion of a pyruvate kinase nucleotide sequence or a NAD⁺-dependent malic enzyme nucleotide sequence. Such pyruvate kinase nucleotide sequences encode cytosolic pyruvate kinases. Cytosolic pyruvate kinase nucleotide sequences include, but are not limited to, EMBL Accession Nos. Z29492 and X53688, GenBank Accession No. L08632 and the nucleotide sequences set forth in SEQ ID NOs: 3 and 5.

In a second embodiment of the invention, the methods of the invention provide a plant, particularly a maize plant, that produces seeds or kernels with an increased level of a desired product including, but not limited to oil, fatty acids, at least one intermediate in lipid biosynthesis, or at least one specialty molecule. Such seeds can have an increased level of the desired product in the tissues of the embryo, endosperm, or both. The specialty molecule can be synthesized in a plant, for example, from an intermediate in fatty acid synthesis. The methods of the invention involve stably incorporating into the genome of a plant at least one nucleotide construct comprising a pyruvate kinase nucleotide sequenceor a malic enzyme nucleotide sequence, operably linked to a promoter that drives expression in a developing seed. Pyruvate kinase nucleotide sequence of the second embodiment can encode a plastidic pyruvate kinases. Such plastidic pyruvate kinase nucleotide sequences include, but are not limited to, the nucleotide sequence set forth in SEQ ID NO: 1. Malic enzyme nucleotide sequences of the second embodiment can encode a NADP⁺-dependent malic enzyme, particularly a plastidic NADP⁺-dependent malic enzyme. The plastidic NADP⁺-dependent malic enzyme nucleotide sequences of the second embodiment include, but are not limited to, GenBank Accession Nos. J05130, AB016804, AW217913, and AI727829. Promoters include, but are not limited to, the oleosin and globulin-1 promoters. In some methods of the invention, both a pyruvate kinase nucleotide sequence and a malic enzyme nucleotide sequences can be introduced into the plant.

If necessary to achieve the desired increase in oil or other desired product, the plant can also be transformed with one or more additional nucleotide constructs. Of particular interest are nucleotide constructs which comprise at least a portion of a nucleotide sequence that encodes an enzyme that catalyzes the synthesis of pyruvate in the cytosol and/or in mitochondria. Such nucleotide constructs can be used to transform a plant to reduce or eliminate the synthesis of pyruvate in the cytosol and in mitochondria. The methods of the invention can additionally involve methods known in the art for reducing or eliminating the activity or level of an enzyme in a cell including, but not limited to antisense suppression, sense suppression, chimeraplasty, and dominant-negative strategies.

To reduce or eliminate the conversion of PEP to pyruvate in the cytosol, the plant can be transformed with a nucleotide construct comprising at least a portion of a nucleotide sequence encoding a cytosolic pyruvate kinase. Reducing or eliminating the conversion of PEP to pyruvate in cytosol can make more malate available for entry into plastids. Any nucleotide sequence encoding a cytosolic pyruvate kinase known in the art can be used in the methods of the invention including, but not limited to, those cytosolic pyruvate kinase nucleotide described supra. Cytosolic pyruvate kinase nucleotide sequences of the invention include, but are not limited to, EMBL Accession Nos. Z29492 and X53688, GenBank Accession No. L08632, and the nucleotide sequences set forth in SEQ ID NOs: 3 and 5.

To reduce or eliminate the conversion of malate to pyruvate in mitochondria, the plant can be transformed with a nucleotide construct comprising at least a portion of an NAD⁺-dependent malic enzyme nucleotide sequence. Malate that is formed in the cytosol, instead of entering the choloroplast, can enter the mitochondria where it can be decarboxylated to form pryuvate, which can then be converted into acetyl-CoA. Acetyl-CoA can then enter the TCA cycle. Reducing or eliminating the decarboxylation of malate in plant mitochondria by antisense suppression of mitochondrial NAD⁺-dependent malic enzyme is known to increase the flux of carbon to plastids in plant storage tissues. See WO 98/23757, herein incorporated by reference.

Any NAD⁺-dependent malic enzyme nucleotide sequence known in the art can be employed in the methods of the present invention to reduce the activity and/or level of NAD⁺-dependent malic enzyme in mitochondria. Two genes encoding different subunits of NAD⁺-dependent malic enzyme have been identified in potato (GenBank Accession Nos. Z23023 and Z23002). Nucleotide sequences encoding either one or both of these subunits can be utilized in the methods of the present invention. NAD⁺-dependent malic enzyme nucleotide sequences of the invention, include but are not limited to, GenBank Accession Nos. BE040183, Z23023, and Z23002.

While the methods of the present invention do not depend on a particular biological mechanism for increasing the oil or other desired product, it is recognized the methods of the present invention can lead to a disruption in the metabolism of the plant leading to an increased flux of carbon flux to the plastids. This increased carbon flux may be the result of an increased rate of transport of malate from the cytosol to the plastids. Other cytosolic metabolites may, however, contribute to the increased flux of carbon to the plastid. Such metabolites include, but are not limited to, 3-phosphoglyceric acid and dihydroxyacetone phosphate.

The invention provides plants, plant tissues, plant cells, and seeds thereof that are genetically modified to alter the synthesis of pyruvate therein. Plants possessing the desired alteration in pyruvate metabolism can be selected by measuring the level of a particular component, or molecule, produced in the plant, or in one or more parts thereof including, but not limited to, seeds, fruits, leaves, stems, roots, flowers, embryos, cotyledons, endosperm, and scutellum. Such components or molecules include, but are not limited to, protein, amino acids, oil, triglycerides, fatty acids, and specialty molecules. The desired components or molecules can be measured by methods known in the art including, but are not limited to, NMR, HPLC, GC, GC-MS, TLC, immunoassays, and the like. If necessary, the desired components or molecules can be extracted from plant tissues using standard extraction techniques that are known in the art.

Compositions of the invention include native nucleotide sequences encoding pyruvate kinases. In particular, the present invention provides for isolated nucleic acid molecules comprising nucleotide sequences encoding the amino acid sequences shown in SEQ ID NOs: 2, 4, and 6, or the nucleotide sequences encoding the DNA sequences deposited in a bacterial host as Patent Deposit No. PTA 2311. Further provided are polypeptides having an amino acid sequence encoded by a nucleic acid molecule described herein, for example those set forth in SEQ ID NOs: 1, 3, and 5, those deposited in a bacterial host as Patent Deposit No. PTA-2311, and fragments and variants thereof.

Plasmids containing the nucleotide sequences of the invention were deposited with the Patent Depository of the American Type Culture Collection (ATCC), Manassas, Va., on Aug. 1, 2000 and assigned Patent Deposit No. PTA-2311. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112.

The invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably culture medium represents less than about 30%, 20%, 10%, or 5% (by dry weight) of chemical precursors or non-protein-of-interest chemicals.

Fragments and variants of the disclosed nucleotide sequences and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence pyruvate kinase activity. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of a pyruvate kinase nucleotide sequence that encodes a biologically active portion of a pyruvate kinase of the invention will encode at least 15, 25, 30, 50, 100, 150, 200, 250, 350, 400, 450, 500 or 550 contiguous amino acids, or up to the total number of amino acids present in a full-length pyruvate kinase of the invention (for example, 569, 509, and 513 amino acids for SEQ ID NOs: 2, 4, and 6, respectively). Fragments of a pyruvate kinase nucleotide sequence that are useful as hybridization probes or PCR primers generally need not encode a biologically active portion of a pyruvate kinase.

Thus, a fragment of a pyruvate kinase nucleotide sequence may encode a biologically active portion of a pyruvate kinase, or it may be a fragment that can be used as a hybridization probe or PCR primer using methods disclosed below. A biologically active portion of a pyruvate kinase can be prepared by isolating a portion of one of the pyruvate kinase nucleotide sequences of the invention, expressing the encoded portion of the pyruvate kinase (e.g., by recombinant expression in vitro), and assessing the activity of the encoded portion of the pyruvate kinase. Nucleic acid molecules that are fragments of a pyruvate kinase nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,600, 1,700 or 1,800 nucleotides, or up to the number of nucleotides present in a full-length pyruvate kinase nucleotide sequence disclosed herein (for example, 2245, 1922, and 2075 nucleotides for SEQ ID NOs: 1, 3, and 5, respectively).

By “variants” is intended substantially similar sequences. For nucleotide sequences, conservative variants include those sequences that, because of the degeneracy of the genetic code, encode the amino acid sequence of one of the pyruvate kinase polypeptides of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode a pyruvate kinase protein of the invention. Generally, variants of a particular nucleotide sequence of the invention will have at least about 65%, 70%, generally at least about 75%, 80%, 85%, preferably at least about 86%, 87%, 88%, 89%, 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, pyruvate kinase activity as described herein. Such variants may result from, for example, genetic polymorphism or from human manipulation. Biologically active variants of a native pyruvate kinase of the invention will have at least about 75%, 80%, generally at least about, 81%, 82, 83%, preferably at least about 84%, 85%, 86%, 87%, 88%, 89%, 90%, 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants of the pyruvate kinases can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferred.

Thus, the genes and nucleotide sequences of the invention include both the naturally occurring sequences as well as mutant forms. Likewise, the proteins of the invention encompass both naturally occurring proteins as well as variations and modified forms thereof. Such variants will continue to possess the desired pyruvate kinase activity. Obviously, the mutations that will be made in the DNA encoding the variant must not place the sequence out of reading frame and preferably will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by pyruvate kinase activity assays. See, for example, Methods of Enzymatic Analysis, Vol. 1 ((1974). Bergmeyer, ed., Verlag Chemie, Weinheim), herein incorporated by reference.

Variant nucleotide sequences and proteins also encompass sequences and proteins derived from a mutagenic and recombinogenic procedure such as DNA shuffling. With such a procedure, one or more different pyruvate kinase coding sequences can be manipulated to create a new pyruvate kinase possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between one or more pyruvate kinase genes of the invention and other known pyruvate kinase genes to obtain a new gene coding for a protein with an improved property of interest, such as an increased Kₘ in the case of an enzyme. Alternatively, two or more of the pyruvate kinase genes of the invention may be shuffled together. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The nucleotide sequences of the invention can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to the entire pyruvate kinase nucleotide sequences set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the pyruvate kinase sequence of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire pryruvate kinase nucleotide sequence disclosed herein, or one or more portions thereof, may be used as a probe capable of specifically hybridizing to corresponding pyruvate kinase nucleotide sequences and messenger RNAs. To achieve specific hybridization under a variety of conditions, such probes include sequences that are unique among pyruvate kinase nucleotide sequences and are preferably at least about 10 nucleotides in length, and most preferably at least about 20 nucleotides in length. Such probes may be used to amplify corresponding pyruvate kinase nucleotide sequences from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least two-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (%GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Thus, isolated sequences that encode a pyruvate kinase and which hybridize under stringent conditions to the pyruvate kinase nucleotide sequences disclosed herein, or to fragments thereof, are encompassed by the present invention.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.hlm.nih.gov. Alignment may also be performed manually by inspection.

Unless otherwise stated, sequence identity/similarity values provided herein refer to the value obtained using GAP Version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The pyruvate kinase nucleotide sequences of the invention are provided in expression cassettes for expression in the plant of interest. The cassette will include 5′ and 3′ regulatory sequences operably linked to a pyruvate kinase sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the pyruvate kinase nucleotide sequence to be under the transcriptional regulation of the regulatory regions. The expression cassette may additionally contain selectable marker genes.

The expression cassette will include in the 5′-3′ direction of transcription, a transcriptional and translational initiation region, a pyruvate kinase DNA sequence of the invention, and a transcriptional and translational termination region functional in plants. The transcriptional initiation region, the promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a chimeric gene comprises a coding sequence operably linked to a transcription initiation region that is heterologous to the coding sequence. That is the transcription initiation region is any transcription initiation region except the native, unmodified transcription initiation region of that coding region.

While it may be preferable to express the sequences using heterologous promoters, the native promoter sequences may be used. Such constructs would change expression levels of pyruvate kinase in the plant or plant cell. Thus, the phenotype of the plant or plant cell is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced pyruvate kinase expression within a particular plant tissue. Tissue-preferred promoters include Yamamoto et al. (1997) Plant J. 12(2)255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2): 207-218 (soybean root-preferred glutamine synthetase gene); Keller and Baumgartner (1991) Plant Cell 3(10):1051-1061 (root-preferred control element in the GRP 1.8 gene of French bean); Sanger et al. (1990) Plant Mol. Biol. 14(3):433-443 (root-preferred promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-preferred promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-preferred promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase); and celA (cellulose synthase) (see the copending application entitled “Seed-Preferred Promoters,” U.S. Application Serial No. 60/097,233, filed Aug. 20, 1998, herein incorporated by reference). Gama-zein is a preferred endosperm-preferred promoter. Glob-1 is a preferred embryo-preferred promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, γ-zein, waxy, shrunken-1, shrunken-2, globulin-1, etc.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci USA 90:1917-1921; Labow et al. (1990) Mol. Cell. Biol. 10:3343-3356; Zambretti et al. (1992) Proc. Natl. Acad. Sci. USA 89:3952-3956; Baim et al (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad Sci USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

In some embodiments of the invention, the proteins encoded by the nucleic acids of interest are targeted to the plastid for expression. In this manner, where the nucleic acid of interest is not directly inserted into the plastid, the expression cassette will additionally contain a nucleic acid encoding a transit peptide to direct the gene product of interest to the plastids. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Plastid-targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The nucleic acids of interest to be targeted to the chloroplast genome may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

It is recognized that with these nucleotide sequences, antisense constructions, complementary to at least a portion of the messenger RNA (mRNA) for the pyruvate kinase sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art and are often referred to as co-suppression methods. The methods generally involve transforming plants with a nucleotide construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See, U.S. Pat. Nos. 5,283,184 and 5,034,323; herein incorporated by reference.

The invention additional encompasses nucleotide sequences encoding variant polypeptides that can be used in dominant-negative strategies to reduce a particular biological activity within an organism or cell thereof. Such dominant-negative strategies are known in the art and can involve the expression of a modified subunit of a multisubunit protein. Generally, such a modified subunit comprises a polypeptide that is able to affect, or interact with, other members of the multisubunit protein complex and thereby reduce, or eliminate, the biological activity of the complex. It is recognized that such dominant-negative strategies can be used to reduce or eliminate the activity of both homomeric enzymes and heteromeric enzymes. By “homomeric enzyme” is intended an enzyme that is comprised of two or more subunits each having the same amino acid sequences. By “heteromeric enzyme” is intended an enzyme that is comprised of two or more subunits wherein not all the subunits comprise the same amino acid sequence. While the methods of the invention do not depend a particular biological mechanism, typically such a dominant-negative approach will involve the expression of a variant of a poplypeptide of the invention that does not possess the complete biological activity of the native polypeptide. It is recognized that such an dominant-negative approach does not depend on eliminating or reducing the expression of native genes in a plant, only that such an approach involves the expression of nucleotide sequence of the invention that encodes a variant polypeptide that is capable of causing a reduction or elimination of the desired biological activity in a plant or cell thereof.

The present invention may be used for transformation of any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Preferably, plants of the present invention are crop plants (for example, corn, soybean, Brassica, wheat, rice, sunflower, cotton, safflower, peanut, sorghum, alfalfa, millet, tobacco, etc.). Plants of particular interest include grain plants that provide seeds of interest, oilseed plants, and leguminous plants. Seeds of interest include grain seeds, such as corn, wheat, barley, rice, sorghum, rye, etc. Oilseed plants include soybean, Brassica, safflower, sunflower, cotton, maize, peanut, alfalfa, palm, sesame, coconut, etc. Leguminous plants include beans and peas. Beans include soybean, guar, locust bean, fenugreek, garden beans, cowpea, mungbean, lima bean, fava bean, lentils, chickpea, etc.

The following examples are presented by way of illustration, not by way of limitation.

### EXPERIMENTAL

### EXAMPLE 1

### Transformation and Regeneration of Transgenic Maize Plants

Immature maize embryos from greenhouse donor plants are bombarded with a plasmid containing a pyruvate kinase or malic enzyme nucleotide sequence operably linked to an oleosin or globulin-1 promoter plus a plasmid containing the selectable marker gene PAT (Wohlleben et al. (1988) Gene 70:25-37) that confers resistance to the herbicide Bialaphos. Transformation is performed as follows. Media recipes follow below.

Preparation of Target Tissue

The ears are surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising a pyruvate kinase or malic enzyme nucleotide sequence operably linked to an oleosin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in TrisEDTA buffer (1 μg total)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for increased protein, oil or a specialty molecule.

Bombardment and Culture Media

Bombardment medium (560Y) comprises 4.0 g/l N6 basal salts (SIGMA C-1416), 1.0 ml/l Eriksson's Vitamin Mix (1000X SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000X SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos(both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### EXAMPLE 2

### Agrobacterium-Mediated Transformation of Maize

For Agrobacterium-mediated transformation of maize with a pyruvate kinase or malic enzyme nucleotide sequence of the invention, preferably the method of Zhao is employed (PCT patent publication WO98/32326), the contents of which are hereby incorporated by reference. Briefly, immature embryos are isolated from maize and the embryos contacted with a suspension of Agrobacterium, where the bacteria are capable of transferring the nucleotide sequence(s) of interest to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

### EXAMPLE 3

### Soybean Embryo Transformation and Rengeneration of Plants

Soybean embryos are bombarded with a plasmid containing a pyruvate kinase or malic enzyme nucleotide sequence operably linked to a seed-preferred promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the pyruvate kinase or malic enzyme nucleotide sequence operably linked to a seed-preferred promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### EXAMPLE 3

### Sunflower Meristem Tissue Transformation and Regeneration of Plants

Sunflower meristem tissues are transformed with an expression cassette comprising a pyruvate kinase or malic enzyme nucleotide sequence operably linked to a seed-preferred promoter as follows (see also European Patent Number EP 0 486233, herein incorporated by reference, and Malone-Schoneberg et al. (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer et al.(1990) Plant Cell Rep. 9: 55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant., 15: 473-497), Shepard's vitamin additions (Shepard (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6, and 8 g/l Phytagar.

The explants are subjected to microprojectile bombardment prior to Agrobacterium treatment (Bidney et al. (1992) Plant Mol. Biol. 18: 301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the pyruvate kinase or malic enzyme nucleotide sequence operably linked to a seed-preferred promoter promoter is introduced into Agrobacterium strain EHA105 via freeze-thawing as described by Holsters et al. (1978) Mol. Gen. Genet. 163:181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for pyruvate kinase activity or malic enzyme activity.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA while transgenic seeds harvested from NPTII-positive T₀ plants are identified by pyruvate kinase activity or malic enzyme activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD 600. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for pyruvate kinase or malic enzyme activity using assays known in the art (see Methods of Enzymatic Analysis, Vol. 1 ((1974). Bergmeyer, ed., Verlag Chemie, Weinheim. After positive (i.e., for a desired level of enzyme activity or protein expression) explants are identified, those shoots that fail to exhibit the desired level of activity or protein are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for the desired protein or activity level are grafted to Pioneer hybrid 6440 in vitro-grown sunflower seedling rootstock. The rootstocks are prepared in the following manner. Seeds are dehulled and surface-sterilized for 20 minutes in a 20% Clorox bleach solution with the addition of two to three drops of Tween 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated nucleotide molecule comprising a nucleotide sequence selected from the group consisting of:
(a) the nucleotide sequence set forth in SEQ ID NO: 1;
(b) the nucleotide sequence set forth in SEQ ID NO: 3;
(c) the nucleotide sequence set forth in SEQ ID NO: 5;
(d) a nucleotide sequence comprising at least 70% sequence identity to the sequence set forth in SEQ ID NO: 1;
(e) a nucleotide sequence comprising at least 75% sequence identity to the sequence set forth in SEQ ID NO: 3;
(f) a nucleotide sequence comprising at least 75% sequence identity to the sequence set forth in SEQ ID NO: 5;
(g) a nucleotide sequence encoding the amino acid sequence set forth in SEQ ID NO: 2;
(h) a nucleotide sequence encoding the amino acid sequence set forth in SEQ ID NO: 4;
(i) a nucleotide sequence encoding the amino acid sequence set forth in SEQ ID NO: 6;
(j) a nucleotide sequence that is complementary to the nucleotide sequence of (a), (b), (c), (d), (e), (f), (g), (h), or (i); and
(k) a nucleotide sequence that hybridizes under stringent conditions to the nucleotide sequence of (a), (b), (c), (d), (e), (f), (g), (h), (i), or (j).

2. An expression cassette comprising the nucleotide molecule of claim 1, wherein said nucleotide sequence is operably linked to a promoter that drives expression in a plant cell.

3. An isolated protein comprising an amino acid sequence selected from the group consisting of:
(a) the amino acid sequence set forth in SEQ ID NO: 2;
(b) the amino acid sequence set forth in SEQ ID NO: 4;
(c) the amino acid sequence set forth in SEQ ID NO: 6;
(d) an amino acid sequence comprising at least 75% sequence identity to the amino acid sequence set forth in SEQ ID NO: 2, wherein said protein comprises pyruvate kinase activity;
(e) an amino acid sequence comprising at least 85% sequence identity to the amino acid sequence set forth in SEQ ID NO: 4, wherein said protein comprises pyruvate kinase activity;
(f) an amino acid sequence comprising at least 90% sequence identity to the amino acid sequence set forth in SEQ ID NO: 6, wherein said protein comprises pyruvate kinase activity;
(g) the amino acid sequence encoded by the nucleotide sequence set forth in SEQ ID NO: 1;
(h) the amino acid sequence encoded by the nucleotide sequence set forth in SEQ ID NO: 3; and
(i) the amino acid sequence encoded by the nucleotide sequence set forth in SEQ ID NO: 5.

4. A transformed plant comprising stably incorporated in its genome a nucleotide construct comprising a nucleotide sequence operably linked to a promoter that drives expression in a plant cell, said nucleotide sequence selected from the group consisting of:
(a) the nucleotide sequence set forth in SEQ ID NO: 1;
(a) the nucleotide sequence set forth in SEQ ID NO: 3;
(b) the nucleotide sequence set forth in SEQ ID NO: 5;
(c) a nucleotide sequence comprising at least 70% sequence identity to the sequence set forth in SEQ ID NO: 1;
(d) a nucleotide sequence comprising at least 75% sequence identity to the sequence set forth in SEQ ID NO: 3;
(e) a nucleotide sequence comprising at least 75% sequence identity to the sequence set forth in SEQ ID NO: 5;
(f) a nucleotide sequence encoding the amino acid sequence set forth in SEQ ID NO: 2;
(g) a nucleotide sequence encoding the amino acid sequence set forth in SEQ ID NO: 4;
(h) a nucleotide sequence encoding the amino acid sequence set forth in SEQ ID NO: 6;
(i) a nucleotide sequence that is complementary to the nucleotide sequence of (a), (b), (c), (d), (e), (f), (g), (h), or (i); and
(j) a nucleotide sequence that hybridizes under stringent conditions to the nucleotide sequence of (a), (b), (c), (d), (e), (f), (g), (h), (i), or (j).

5. The plant of claim 4, wherein said promoter is selected from the group consisting of tissue-preferred, chemical-regulated, and constitutive promoters.

6. The plant of claim 5, wherein said tissue-preferred promoter is selected from the group consisting of seed-preferred, embryo-preferred, and endosperm-preferred promoters.

7. The plant of claim 4, wherein said plant is a monocot.

8. The plant of claim 7, wherein said monocot is selected from the group consisting of maize, wheat, rice, millet, sorghum, barley, and rye.

9. The plant of claim 4, wherein said plant is a dicot.

10. The plant of claim 9, wherein said dicot is selected from the group consisting of soybean, Brassica sp., alfalfa, safflower, sunflower, peanut, cotton, and potato.

11. Transformed seed of the plant of claim 4.

12. A method for increasing protein in a plant comprising introducing into a plant a nucleotide construct comprising a nucleotide sequence operably linked to a promoter that drives expression in a plant cell, said nucleotide sequence selected from the group consisting of:
(a) the nucleotide sequence set forth in SEQ ID NO: 1;
(b) the nucleotide sequence set forth in SEQ ID NO: 3;
(c) the nucleotide sequence set forth in SEQ ID NO: 5;
(d) a nucleotide sequence comprising at least 70% sequence identity to the sequence set forth in SEQ ID NO: 1;
(e) a nucleotide sequence comprising at least 75% sequence identity to the sequence set forth in SEQ ID NO: 3;
(f) a nucleotide sequence comprising at least 75% sequence identity to the sequence set forth in SEQ ID NO: 5;
(g) a nucleotide sequence encoding the amino acid sequence set forth in SEQ ID NO: 2;
(h) a nucleotide sequence encoding the amino acid sequence set forth in SEQ ID NO: 4;
(i) a nucleotide sequence encoding the amino acid sequence set forth in SEQ ID NO: 6;
(j) a nucleotide sequence that is complementary to the nucleotide sequence of (a), (b), (c), (d), (e), (f), (g), (h), or (i); and
(k) a nucleotide sequence that hybridizes under stringent conditions to the nucleotide sequence of (a), (b), (c), (d), (e), (f), (g), (h), (i), or (j);
wherein the level of protein is increased in said plant or at least one part thereof.

13. The method of claim 12, wherein said part is a seed or a fruit.

14. The method of claim 12, wherein said part is an embryo of a seed.

15. The method of claim 12, wherein said part is the endosperm of a seed.

16. A method for increasing oil or other desired product in a plant comprising introducing into a plant at least one nucleotide construct comprising a nucleotide sequence operably linked to a promoter that drives expression in a plant cell, wherein said nucleotide sequence is a pyruvate kinase nucleotide sequence or an NADP⁺-dependent malic enzyme nucleotide sequence, and wherein said oil or other desired product is increased in said plant or at least one part thereof;
wherein said nucleotide sequence encoding NADP⁺-dependent malic enzyme is selected from the group consisting of GenBank Accession Nos. J05130, AB016804, AW217913, and AI727829; and
wherein said nucleotide sequence encoding pyruvate kinase is selected from the group consisting of:
(a) a nucleotide sequence encoding a maize pyruvate kinase;
(b) a nucleotide sequence encoding a plastidic pyruvate kinase
(c) the nucleotide sequence set forth in SEQ ID NO: 1;
(d) a nucleotide sequence comprising at least 70% sequence identity to the sequence set forth in SEQ ID NO: 1;
(e) a nucleotide sequence encoding the amino acid sequence set forth in SEQ ID NO: 2;
(f) a nucleotide sequence encoding an amino acid sequence comprising at least 75% sequence identity to the sequence set forth in SEQ ID NO: 2;
(g) a nucleotide sequence that is complementary to the nucleotide sequence of (a), (b), (c), (d), (e), or (f); and
(h) a nucleotide sequence that hybridizes under stringent conditions to the nucleotide sequence of (a), (b), (c), (d), (e), (f), or (g).

17. The method of claim 16, wherein said desired product is selected from the group consisting of oil, a triglyceride, an intermediate in oil synthesis, a fatty acid, and a specialty molecule.

18. The plant of claim 16, wherein said part is a seed or a fruit.

19. The plant of claim 16, wherein said part is the embryo of a seed.

20. The plant of claim 16, wherein said part is the endosperm of a seed.

21. A plant genetically manipulated for increased synthesis of oil or other desired product, said plant comprising in its genome:
(a) a first nucleotide construct comprising a NADP⁺-dependent malic enzyme nucleotide sequence operably linked to a first promoter that drives expression in a plant cell; and
(b) a second nucleotide construct comprising a pyruvate kinase nucleotide sequence operably linked to a second promoter that drives expression in a plant cell;
wherein the level of said oil or said desired product is increased in said plant or at least one part thereof.

22. The plant of claim 21, wherein said NADP⁺-dependent malic enzyme nucleotide sequence is selected from the group consisting of GenBank Accession Nos. J05130, AB016804, AW217913, and AI727829.

23. The plant of claim 21, wherein said pyruvate kinase nucleotide sequence encoding is selected from the group consisting of:
(a) the nucleotide sequence encoding a maize pyruvate kinase;
(b) the nucleotide sequence encoding a plastidic pyruvate kinase
(c) the nucleotide sequence set forth in SEQ ID NO: 1;
(d) a nucleotide sequence comprising at least 70% sequence identity to the sequence set forth in SEQ ID NO: 1;
(e) a nucleotide sequence encoding the amino acid sequence set forth in SEQ ID NO: 2;
(f) a nucleotide sequence encoding an amino acid sequence comprising at least 75% sequence identity to the sequence set forth in SEQ ID NO: 2;
(g) a nucleotide sequence that is complementary to the nucleotide sequence of (a), (b), (c), (d), (e), or (f); and
(h) a nucleotide sequence that hybridizes under stringent conditions to the nucleotide sequence of (a), (b), (c), (d), (e), (f), or (g).

24. The plant of claim 21, wherein said desired product is selected from the group consisting of oil, a triglyceride, an intermediate in oil synthesis, a fatty acid, and a specialty molecule.

25. The plant of claim 21, wherein at least one of said first and said second nucleotide constructs further comprises an operably linked nucleotide sequence encoding a plastid transit peptide.

26. Transformed seed of the plant of claim 21.
